(19) United States
(12) Patent Application Publication
(10) Pub. No.: US 2008/0021465 A1
(43) Pub. Date: Jan. 24, 2008

(54) SPINE TREATMENT DEVICES AND METHODS

(76) Inventors: John H. Shadduck, Tiburon, CA (US); Csaba Truckai, Saratoga, CA (US); Robert Luzzi, Pleasanton, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/780,967

(22) Filed: Jul. 20, 2007

Related U.S. Application Data
(60) Provisional application No. 60/832,121, filed on Jul. 21, 2006.
(60) Provisional application No. 60/831,925, filed on Jul. 20, 2006.

(51) Int. Cl. A61B 17/58; A61B 17/56

(52) U.S. Cl. 606/61; 606/100; 606/73

(57) ABSTRACT

A spine implant device for fusion or dynamic stabilization of a spine segment can include a fixation device with a shaft portion for engaging bone and a proximal end for coupling to a rod that allows for limited flexing of the proximal end relative to the shaft portion. A spine implant system and method utilizing such a bone fixation device can include an energy delivery mechanism for on-demand curing of bone cement delivered through the bone fixation device into a bone.

### CROSS-REFERENCE TO RELATED APPLICATIONS

This application claims the benefit of U.S. Provisional Patent Application No. 60/832,121 filed Jul. 21, 2006, and of U.S. Provisional Patent Application No. 60/831,925, filed Jul. 20, 2006, the entire contents of both of which are incorporated herein by reference and should be considered a part of this specification.

### BACKGROUND OF THE INVENTION

1. Field of the Invention

The invention relates generally to implant systems and methods for treating a spine disorder, and more particularly relates to bone fixation devices and systems configured for fusion and dynamic stabilization systems for re-distributing loads within a spine segment while still allowing for flexion, extension, lateral bending and torsion.

2. Description of the Related Art

Thoracic and lumbar spinal disorders and discogenic pain are major socio-economic concerns in the United States affecting over 70% of the population at some point in life. Low back pain is the most common musculoskeletal complaint requiring medical attention; it is the fifth most common reason for all physician visits. The annual prevalence of low back pain ranges from 15% to 45% and is the most common activity-limiting disorder in persons under the age of 45.

Degenerative changes in the intervertebral disc often play a role in the etiology of low back pain. Many surgical and non-surgical treatments exist for patients with degenerative disc disease (DDD), but often the outcome and efficacy of these treatments are uncertain. In current practice, when a patient has intractable back pain, the physician's first approach is conservative treatment with the use of pain killing pharmacological agents, bed rest and limiting spinal segment motion. Only after an extended period of conservative treatment will the physician consider a surgical solution, which often is spinal fusion of the painful vertebral motion segment. Fusion procedures are highly invasive procedure that carries surgical risk as well as the risk of transition syndrome described above wherein adjacent levels will be at increased risk for facet and discogenic pain.

More than 150,000 lumbar and nearly 200,000 cervical spinal fusions are performed each year to treat common spinal conditions such as degenerative disc disease and spondylolisthesis, or misaligned vertebrae. Some 28 percent are multi-level, meaning that two or three vertebrae are fused. Such fusions “weld” unstable vertebrae together to eliminate pain caused by their movement. While there have been significant advances in spinal fusion devices and surgical techniques, the procedure does not always work reliably. In one survey, the average clinical success rate for pain reduction was about 75%; and long time intervals were required for healing and recuperation (3-24 months, average 15 months). Probably the most significant drawback of spinal fusion is termed the “transition syndrome” which describes the premature degeneration of discs at adjacent levels of the spine. This is certainly the most vexing problem facing relatively young patients when considering spinal fusion surgery.

Many spine experts consider the facet joints to be the most common source of spinal pain. Each vertebra possesses two sets of facet joints, one set for articulating to the vertebra above and one set for the articulation to the vertebra below. In association with the intervertebral discs, the facet joints allow for movement between the vertebrae of the spine. The facet joints are under a constant load from the weight of the body and are involved in guiding general motion and preventing extreme motions in the trunk. Repetitive or excessive trunkal motions, especially in rotation or extension, can irritate and injury facet joints or their encasing fibers. Also, abnormal spinal biomechanics and bad posture can significantly increase stresses and thus accelerate wear and tear on the facet joints.

Recently, technologies have been proposed or developed for disc replacement that may replace, in part, the role of spinal fusion. The principal advantage proposed by complete artificial discs is that vertebral motion segments will retain some degree of motion at the disc space that otherwise would be immobilized in more conventional spinal fusion techniques. Artificial facet joints are also being developed. Many of these technologies are in clinical trials. However, such disc replacement procedures are still highly invasive procedures, which require an anterior surgical approach through the abdomen.

Clinical stability in the spine can be defined as the ability of the spine under physiologic loads to limit patterns of displacement so as to not damage or irritate the spinal cord or nerve roots. In addition, such clinical stability will prevent incapacitating deformities or pain due to later spine structural changes. Any disruption of the components that stabilized a vertebral segment (e.g., disc, facets, ligaments) decreases the clinical stability of the spine.

Improved devices and methods are needed for treating dysfunctional intervertebral discs and facet joints to provide clinical stability, in particular: (i) implantable devices that can be introduced to offset vertebral loading to treat disc degenerative disease and facets through least invasive procedures; (ii) implants and systems that can restore disc height and foraminal spacing; and (iii) implants and systems that can re-distribute loads in spine flexion, extension, lateral bending and torsion.

### SUMMARY OF THE INVENTION

In accordance with one embodiment, a bone implant device is provided. The bone implant device comprises a body configured for implantation in a bone, the body having a proximal body portion and an elongated shaft portion having a surface engageable with the bone, and a resilient body disposed intermediate the proximal body portion and the shaft portion, the resilient body configured to allow the proximal body portion and the shaft portion relative to move relative to each other.

In accordance with another embodiment, a bone implant device is provided, comprising a body configured for implantation into a vertebra, the body having a proximal body portion and a shaft portion defining a flow passageway therethrough. The flow passageway is in communication with at least one outlet port formed on the shaft portion, the flow passageway configured for delivering a flow of bone cement therethrough into the vertebra to substantially fix the bone implant device thereto. The proximal body portion comprises an electrical connector removably coupleable to an electrical source.

In accordance with still another embodiment, a system for treating a spine motion segment is provided. The system comprises a plurality of transpedicular bone implant devices, each implant device having a proximal body portion and a shaft body portion defining a flow passageway therethrough in communication with at least one outlet port formed on the shaft portion, the flow passageway configured for delivering a bone cement flow therethrough into the spine segment. The system also comprises a rod removably coupleable to the plurality of bone implant devices, the rod comprising at least one electrical connector coupleable to an electrical source, the rod being actuatable by the electrical source to alter a physical characteristic of the rod.

In accordance with yet another embodiment, a method for treating a spine segment is provided. The method comprises inserting a plurality of bone implant devices through an incision in a patient, fixating the bone implant devices to at least one vertebra of the spine segment, coupling an extension member between the bone implant devices, and actuating the rod via an electrical source to change the rod from a flexible configuration to a rigid configuration

### BRIEF DESCRIPTION OF THE DRAWINGS

The features and advantages of this invention, and the manner of attaining them, will become apparent by reference to the following description of preferred embodiments of the invention taken in conjunction with the accompanying drawings, wherein:

FIG. 1 is a schematic perspective view of a bone fixation device, in accordance with one embodiment having a flexible portion.

FIG. 2 is a schematic cross-sectional view of the device of FIG. 1 in a repose position.

FIG. 3 is a schematic cross-sectional view of the device of FIGS. 1-2 in a stressed or flexed position.

FIG. 4A is a schematic cross-sectional view of another embodiment of a bone fixation device.

FIG. 4B is a schematic cross-sectional view of another embodiment of a bone fixation device similar to the device in FIG. 4A.

FIG. 5 is a schematic perspective view of still another embodiment of an implant device.

FIG. 6 is a schematic perspective view of yet another embodiment of an implant device.

FIG. 7 is a schematic perspective view of another embodiment of an implant device.

### DETAILED DESCRIPTION OF THE PREFERRED EMBODIMENT

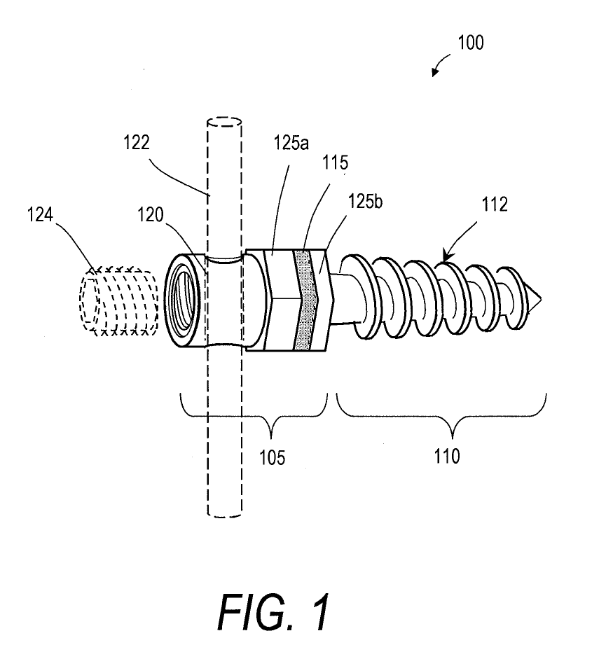

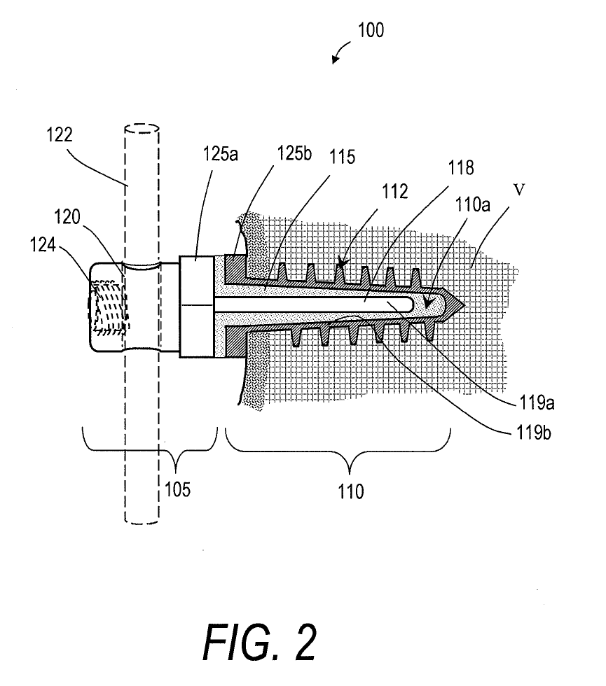

FIGS. 1 and 2 illustrate one embodiment of a bone fixation device 100 that can be used in a rod and pedicle screw system for spine stabilization, either fusion or dynamic. The device has a first end portion or head portion 105 and a second end portion or shaft portion 110 that can be introduced into a bore in a vertebra or bone V (see FIG. 2). In one embodiment, the single or multiple-flight threads 112 are preferably of a self-tapping type. As shown in FIG. 2, a resilient polymer member 115 can be disposed intermediate the head portion 105 and the shaft portion 110, and within a cavity 110a of the shaft portion 110. In the illustrated embodiment, the head portion 105 can be coupled to an elongated element 118 that extends axially within an elongated region of the resilient polymer member 115. A surface 119a of the elongated element 118 and a surface 119b of the shaft portion 110 can have any suitable texture or features for adherence to the polymer 115. The head portion 105 and shaft portion 110 of the bone fixation device 100 can be made of any suitable material used in spinal implants, including a metal.

Still referring to FIGS. 1 and 2, the head portion 105 of the bone fixation device 100 can have a bore 120 therein for receiving a rod or connecting element 122, to which a plurality of such fixation devices 100 can be coupled. The rod element 122 can be secured in head portion 105 by any suitable mechanism, such as a set screw 124. However, other rod-to-screw locking systems are possible, such as (i) a thermally actuated polymer rod 122 (or head portion 105) that changes in cross-section after energy is delivery thereto from an energy source so as to expand the rod 122 in the bore 120; (ii) a shape memory alloy in the rod 122 or head portion 105 that is actuatable by a resistive heater between temporary and memory cross-sections to expand the rod 122 in the bore 120, or vice versa, or (iii) an osmotic material or polymer in the rod 122 or head portion 105 for similarly causing an interference fit between the rod 122 and the bore 120. In FIGS. 1-3, it can be seen that the bone fixation device 100 can have polygonal driving surface portions 125a and 125b on the respective head portion 105 and shaft portion 110, which can be engaged by a driver to rotate the head portion 105 and/or shaft portion 110.

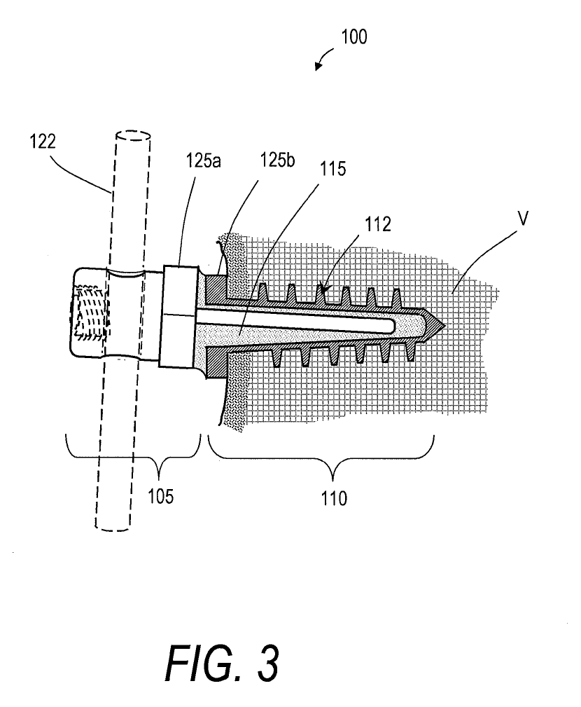

FIG. 3 depicts the bone fixation device 100 of FIG. 1 in use, where the head portion 105 is capable of flexing somewhat relative to the shaft portion 110 that is fixated in the bone. Such flexing can occur, for example, during motion of the spine (e.g., in flexion, torsion, lateral bending and extension).

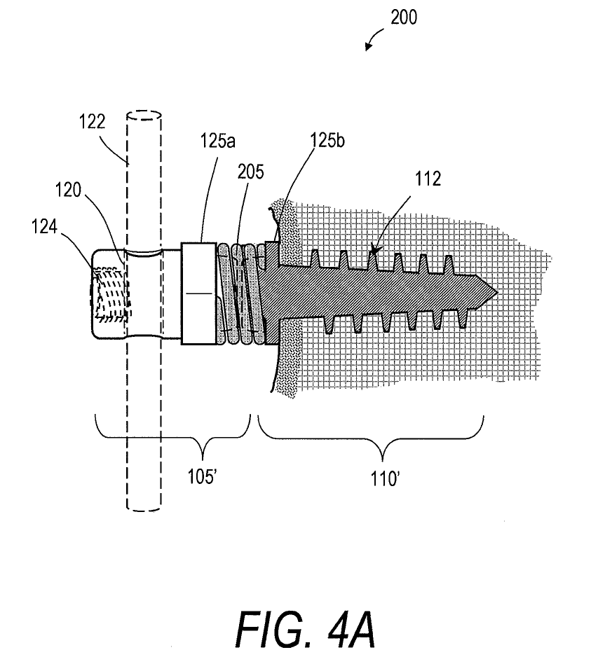

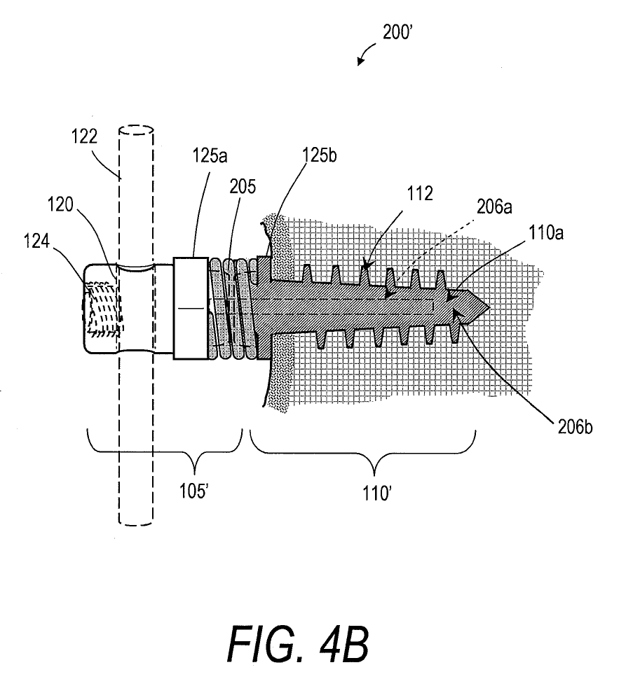

FIG. 4A depicts another embodiment of a bone fixation device 200. The bone fixation device 200 is similar to the bone fixation device 100 discussed above in connection with FIGS. 1-3. Thus, the reference numerals used to designate corresponding components in the bone fixation device 200 and the bone fixation device 100 are identical, except that a “′” is added where there are differences.

In the illustrated embodiment, the bone fixation device 200 has a head portion 105′ and a shaft portion 110′ that are each made of metal and coupled (e.g. welded) to an intermediate helical spring 205. In the illustrated embodiment, the shaft 110′ is a solid shaft. The head portion 105′ and shaft portion 110′ can each have extension portions (shown in phantom in FIG. 4A) that can protrude into the cylindrical space defined by the spring 205. In another embodiment, the spring 205 can comprise a shape memory alloy.

In another embodiment shown in FIG. 4B, which is similar to the bone fixation device 200 and for which similar components are identified with identical reference numerals, a bone fixation device 200′ can include an elongated member 206a (shown in phantom) attached to the head portion 105′ and extending into a cavity 110a′ of the shaft member 110′ filled with a polymer material 206b. The elongated member 206a can extend through a port in the extension portion at the proximal end of the shaft member 110′ and into the cavity 111a′. In both the bone fixation devices 200, 200′, the head portion 105′ can flex in a similar manner to that described above in connection with the embodiment of FIG. 3.

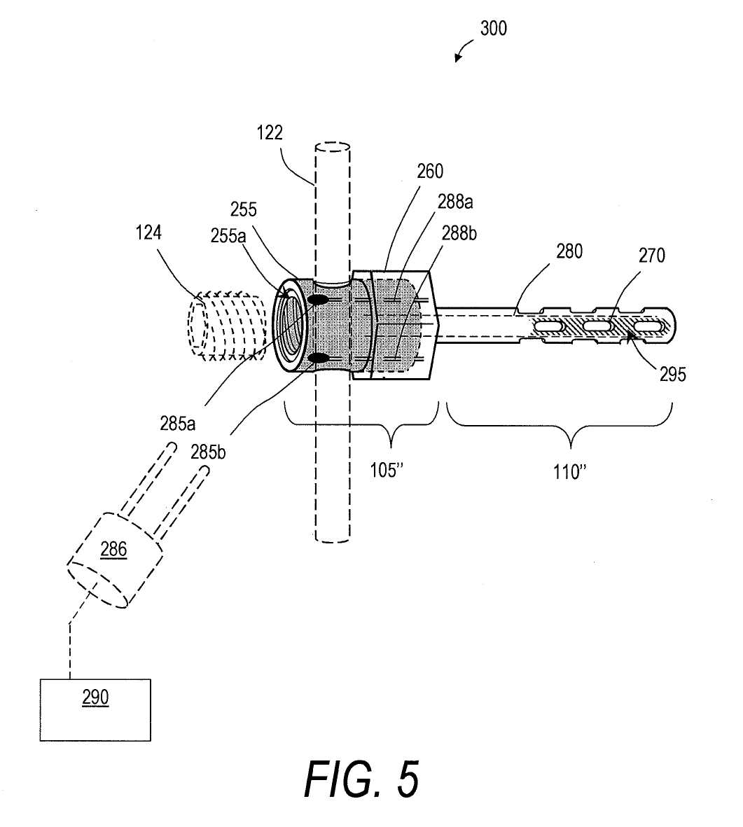

FIG. 5 depicts another embodiment of a bone fixation device 300. The bone fixation device 300 is similar to the bone fixation device 100 discussed above in connection with FIGS. 1-3. Thus, the reference numerals used to designate corresponding components in the bone fixation device 300 and the bone fixation device 100 are identical, except that a “″” is added where there are differences.

In the illustrated embodiment, the bone fixation device 300 has a head portion 105″ that can flex relative to a shaft portion 110″. In this embodiment, the head portion 105″ includes a resilient polymer 255 that can be provided (e.g., locked) in a proximal end 260 of the shaft portion 110″. The resilient polymer 255 can have a threaded metal sleeve 255a therein for receiving the set screw 124, and optionally have a metal sleeve about the rod 122.

With continued reference to FIG. 5, the shaft portion 110″ of the bone fixation device 300 can have a bore 280 therethrough for delivering a bone cement therethrough and through at least one end or side port 270 for fixing the bone fixation device 300 in bone. Thus, the bone fixation device 300 need not be threaded as shown in the embodiments of FIGS. 1-4. Further, the bone fixation device 300 can have an electrical connector or connection mechanism, including at least one electrical lead (opposing polarity connectors 285a and 285b in FIG. 5) in a proximal end 260 of the device 300 for connecting with a releaseable electrical connector 286 that connects to an electrical source 290. In one embodiment, opposing polarity leads 288a, 288b extend to opposing polarity electrodes 285a, 285b, respectively, and/or to a heating element 295, which can be disposed in a distal portion of the shaft portion 110″ within the interior bore 280. In one embodiment, the heating element can be a thermal energy emitter. In other embodiments, the heating element can be a resistive heating element, an electrical source, an electrode, a light energy source, an ultrasound source or a microwave source. In another embodiment, the heating element can be disposed on the exterior of the shaft portion 110″.

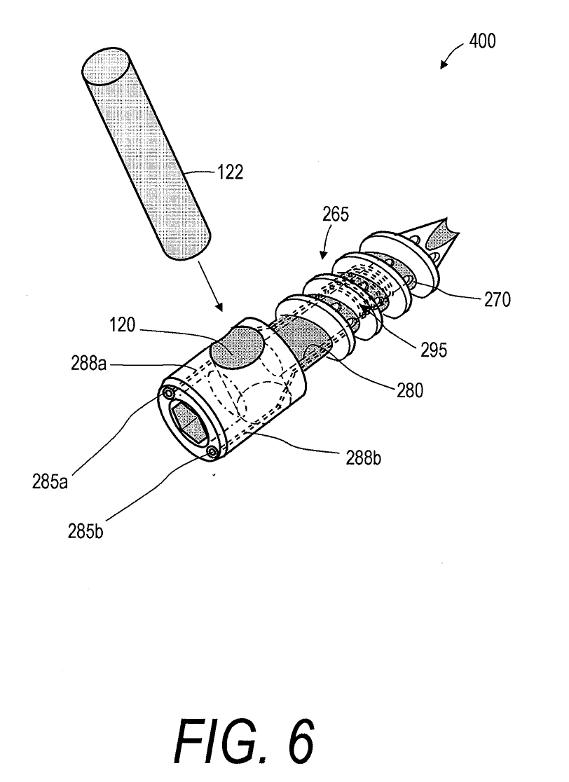

FIG. 6 depicts another embodiment of a bone fixation device 400. The bone fixation device 400 is similar to the bone fixation device 300 discussed above in connection with FIG. 5. Thus, the reference numerals used to designate corresponding components in the bone fixation device 400 and the bone fixation device 400 are identical.

In the illustrated embodiment, the bone fixation device 400 has a threaded shaft portion or axially-extending member 265 that includes a bore 280 therethrough for delivering a bone cement therethrough and through at least one end or side port 270 to thereby fix the bone fixation device 400 in bone. The bone fixation device 400 has an electrical connector or connection mechanism including at least one electrical lead (opposing polarity connectors 285a and 285b in FIGS. 5-6) in a proximal end of the bone fixation device 400 for cooperating with a releaseable electrical connector, such as the electrical connector 286 shown in FIG. 5, connected to an electrical source, such as the electrical source 290 in FIG. 5. In the illustrated embodiment, the opposing polarity connectors 285a, 285b can extend to opposing polarity leads 288a, 288b, which can extend to a resistive heating element 295 in a distal portion of, and within the interior bore 280, of the shaft 265. In another embodiment, the resistive heating element can be disposed on an exterior surface of the shaft 265.

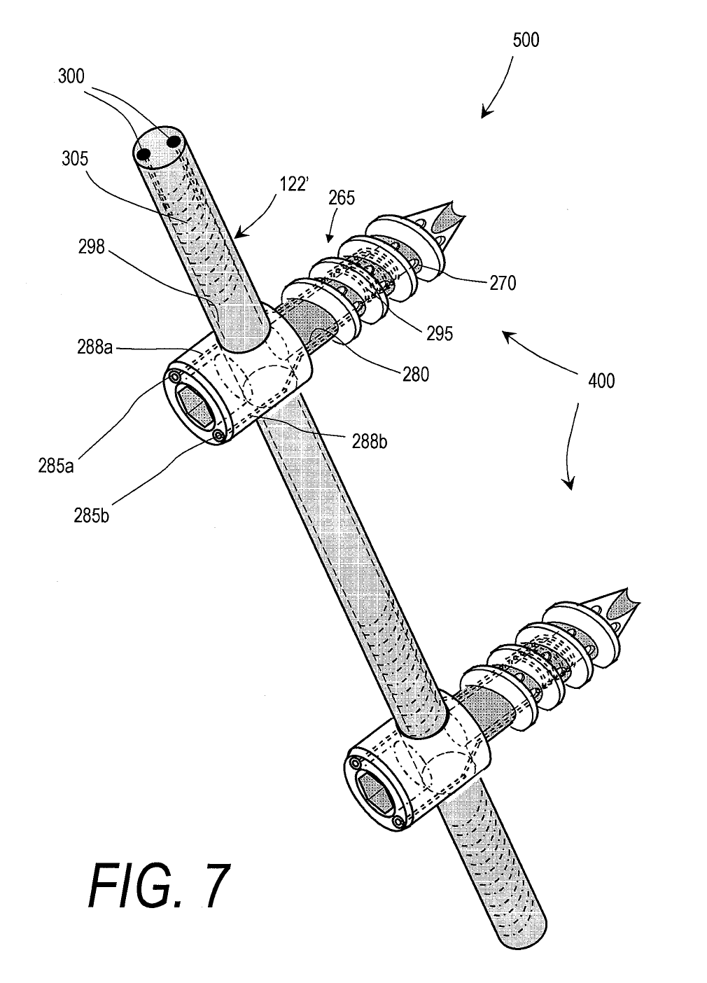

FIG. 7 illustrates one embodiment of spine treatment system 500 wherein the rod member 122′ is actuatable by an energy source. In one embodiment, the rod can be a fluid-tight polymer sleeve with a lumen 298 therein that carries (i) a partly polymerized fluid, gel or paste; (ii) a mixture of a polymer particles and separate monomer for mixing to harden the polymer, or (iii) any other two-part in-situ hardenable polymeric composition. The rod 122′ can include at least one electrical connector 300 for coupling an electrical source, such as the electrical source 290 in FIG. 5, to the rod 122′.

In one embodiment, the electrical source can be actuated to heat at least one resistive coil 305 that extends along at least a portion of the length of the sleeve or conductive resistively heatable polymer portion. The heating of the polymer can be utilized to cause the polymeric portion to change the rod 122′ from flexible to rigid via polymerization of the composition, or to alter the modulus of the rod 122′. In one embodiment a polymeric heating element is used, and the polymer can comprise a PTC (positive temperature coefficient) material to limit heating of the polymer. In another embodiment, the electrical system (e.g., electrical connectors 286, 300 and electrical source 290) also can be used to swell the rod 122′ so as to cause an interference fit between the rod 122′ and a bore in the bone fixation device(s) to lock the rod 122′ to the bone fixation device(s). Though the illustrated embodiment shows the rod 122′ coupled to two bone fixation devices 400, one of ordinary skill in the are will recognize that the rod 122′ can be coupled to any number of bone fixation devices 400, and to any embodiment of bone fixation device disclosed herein, such as bone fixation devices, 100, 200, and 300.

The bone fixation device 100, 200, 300, 400 can include any suitable material used in spinal implants, including a metal, metal alloy and a polymer.

Certain embodiments described above provide new ranges of minimally invasive, reversible treatments that from a new category between traditional conservative therapies and the more invasive surgeries such as fusion procedures or disc replacement procedures.

Certain embodiments include implant system that can be implanted in a very minimally invasive procedure, and requires only small bilateral incisions in a posterior approach. A posterior approach would be highly advantageous for patient recovery. Of particular interest, the inventive procedures are “modular” in that separate implant components are used that can be implanted in a single surgery or in sequential interventions. Certain embodiments of the inventive procedures are for the first time reversible, unlike fusion and disc replacement procedures. Additionally, embodiments of the invention include implant systems that can be partly or entirely removable. Further, in one embodiment the system allows for in-situ adjustment requiring, for example, a needle-like penetration to access the implant.

In certain embodiments, the implant system can be considered for use far in advance of more invasive fusion or disc replacement procedures. In certain embodiments, the inventive system allows for dynamic stabilization of a spine segment in a manner that is comparable to complete disc replacement. Embodiments of the implant system are configured to improve on disc replacement in that it can augment vertebral spacing (e.g., disc height) and foraminal spacing at the same time as controllably reducing loads on facet joints—which complete disc replacement may not address. Certain embodiments of the implant systems are based on principles of a native spine segment by creating stability with a tripod load receiving arrangement. The implant arrangement thus supplements the spine's natural tripod load-bearing system (e.g., disc and two facet joints) and can re-distribute loads with the spine segment in spine torsion, extension, lateral bending and flexion.

Of particular interest, since the embodiments of implant systems are far less invasive than artificial discs and the like, the systems likely will allow for a rapid regulatory approval path when compared to the more invasive artificial disc procedures.

Other implant systems and methods within the spirit and scope of the invention can be used to increase intervertebral spacing, increase the volume of the spinal canal and off-load the facet joints to thereby reduce compression on nerves and vessels to alleviate pain associated therewith.

Although these inventions have been disclosed in the context of a certain preferred embodiments and examples, it will be understood by those skilled in the art that the present inventions extend beyond the specifically disclosed embodiments to other alternative embodiments and/or uses of the inventions and obvious modifications and equivalents thereof. For example, any of the implants disclosed above can be made of a metal material, polymer material, a shape memory alloy, or any suitable material for use in spinal implants. In addition, while a number of variations of the inventions have been shown and described in detail, other modifications, which are within the scope of the inventions, will be readily apparent to those of skill in the art based upon this disclosure. It is also contemplated that various combinations or subcombinations of the specific features and aspects of the embodiments may be made and still fall within one or more of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above. Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Further variations will be apparent to one skilled in the art in light of this disclosure and are intended to fall within the scope of the appended claims.

What is claimed is:

1. A bone implant device, comprising:
a body configured for implantation in a bone, the body having a proximal body portion and an elongated shaft portion having a surface engageable with the bone; and
a resilient body disposed intermediate the proximal body portion and the shaft portion, the resilient body configured to allow the proximal body portion and the shaft portion relative to move relative to each other.

2. The bone implant device of claim 1, wherein the resilient body comprises a polymer.

3. The bone implant device of claim 2, wherein the proximal body portion comprises an elongated element extending axially within the resilient body, the resilient body at least partially disposed in a cavity of the shaft portion.

4. The bone implant device of claim 1, wherein the resilient body is a helical spring coupled to the proximal body portion and shaft portion

5. The bone implant device of claim 4, wherein the spring is welded to the proximal body portion and shaft portion.

6. The bone implant device of claim 4, wherein the spring comprises a shape memory alloy

7. The bone implant device of claim 1, wherein the surface of the shaft portion comprises a plurality of threads.

8. The bone implant device of claim 1, wherein the proximal body portion defines a bore configured to receive an elongated connecting element therethrough.

9. A bone implant device, comprising:
a body configured for implantation into a vertebra, the body having a proximal body portion and a shaft portion defining a flow passageway therethrough, the flow passageway being in communication with at least one outlet port formed on the shaft portion, the flow passageway configured for delivering a flow of bone cement therethrough into the vertebra to substantially fix the bone implant device thereto, the proximal body portion comprising an electrical connector removably coupleable to an electrical source.

10. The bone implant device of claim 9, further comprising a heating element disposed in the shaft portion, the heating element electrically connected to the electrical connector and configured to apply thermal energy to the bone cement prior to ejection thereof into the vertebra.

11. The bone implant device of claim 10, wherein the heating element comprises a resistive heating element.

12. The bone implant device of claim 9, wherein the proximal body portion comprises a head portion comprising a resilient polymer, the head portion configured to flex relative to the shaft portion

13. The bone implant device of claim 12, further comprising a threaded sleeve disposed in the head portion, the threaded sleeve configured to receive a locking member therein to removably lock a rod extending through a bore in the head portion

14. The bone implant device of claim 9, wherein the shaft portion is threadably coupleable to the vertebra.

15. A system for treating a spine motion segment, the system comprising:
a plurality of transpedicular bone implant devices, each implant device having a proximal body portion and a shaft body portion defining a flow passageway therethrough in communication with at least one outlet port formed on the shaft portion, the flow passageway configured for delivering a bone cement flow therethrough into the spine segment; and
a rod removably coupleable to the plurality of bone implant devices, the rod comprising at least one electrical connector coupleable to an electrical source, the rod being actuatable by the electrical source to alter a physical characteristic of the rod.

16. The system of claim 15, wherein the rod is a fluid-tight polymer sleeve comprises a lumen therein.

17. The system of claim 16, wherein the lumen carries a two-part in-situ hardenable polymeric composition.

18. The system of claim 16, wherein the lumen carries a composition chosen from the group consisting of: a partly polymerized fluid, gel, paste, and a mixture of polymer particles and a separate monomer configured to harden the polymer upon mixing.

19. The system of claim 16, wherein the rod comprises at least one resistive heater extending along at least a portion of the length of the sleeve, the resistive heater actuatable to heat the polymer to change the rod from a flexible configuration to a rigid configuration via polymerization of the polymer.

20. The system of claim 19, wherein the resistive heater comprises a positive temperature coefficient material configured to limit the heating of the polymer.

21. A method for treating a spine segment, comprising:
inserting a plurality of bone implant devices through an incision in a patient;
fixating the bone implant devices to at least one vertebra of the spine segment;
coupling an extension member between the bone implant devices; and
actuating the rod via an electrical source to change the rod from a flexible configuration to a rigid configuration.

22. The method of claim 21, wherein fixating the bone implant device comprises flowing a bone cement through the bone implant device and heating the bone cement prior to ejection thereof from the bone implant device into the vertebra.

23. The method of claim 21, wherein actuating the rod comprises heating a polymer within the rod to polymerize the polymer.

24. The method of claim 21, wherein actuating the rod comprises heating the rod to alter a cross-sectional dimension of the rod.
